(19) United States
(12) Patent Application Publication
(10) Pub. No.: US 2023/0158241 A1
(43) Pub. Date: May 25, 2023

(54) NEEDLE-FREE INJECTION SYSTEM

(71) Applicants: FENG-JU CHUANG, TAOYUAN COUNTY (TW); BRIGHTON PRECISION ENGINEERING CO., LTD., TAOYUAN COUNTY (TW)

(72) Inventor: FENG-JU CHUANG, TAOYUAN COUNTY (TW)

(21) Appl. No.: 18/058,815

(22) Filed: Nov. 25, 2022

(30) Foreign Application Priority Data
Nov. 25, 2021 (TW) 110144048
Nov. 25, 2021 (TW) 110214033
Dec. 20, 2021 (TW) 110215138
Jun. 2, 2022 (TW) 111120710

(51) Int. Cl. A61M 5/30; A61M 5/20

(52) U.S. Cl. CPC A61M 5/30; A61M 5/2046; A61M 2205/502

(57) ABSTRACT

The invention provides a needle-free injection system, which mainly includes a pressure accumulator, an output pipe, a delivery device and a fluid supply device. The front end of the pressure accumulator is connected to the output pipe, and the bottom end of the pressure accumulator can be connected to the fluid supply device and/or a hand-holding device. A control panel and a shooting button are fixed on the outer surface of the pressure accumulator. The control panel is electrically connected to the delivery device, and can store a delivery program to control the delivery steps of a delivery material stored in the delivery device, such as the delivery time, delivery quantity or delivery times of the delivery material into the output pipe. In this way, not only the convenience of use can be improved, but also the use space of the needle-free injection system can be reduced.

### REFERENCE TO RELATED APPLICATIONS

This non-provisional application claims priority claim under 35 U.S.C. § 119(a) on Taiwan Patent Application No. 110144048 (filed Nov. 25, 2021), No. 110214033 (filed Nov. 25, 2021), No. 110215138 (filed Dec. 20, 2021) and No. 111120710 (filed Jun. 2, 2022), the entire contents of which are incorporated herein by reference.

### FIELD OF THE INVENTION

The present invention relates to a needle-free injection system, which can inject a delivery material into the cells of a target object by a high-pressure gas, thereby reducing discomfort and injection risks caused by traditional intramuscular injection needles.

### BACKGROUND

A needle-free injection system, such as a gene gun, is a gene transfer device. The needle-free injection system mainly dissolves a delivery material in a solution or adheres to the surface of a metal particle, such as tungsten or gold powder, and then uses a high-pressure gas to accelerate the solution or metal particle, so that the delivery material can be injected in a cell body (target object) such as a skin tissue, a cell wall or a cell membrane. The delivery material can be a medicine, a biological substance, a DNA or an RNA. The needle-free injection system is widely used in the fields of gene transfer of animals or plants, DNA vaccines, gene therapy or aesthetic medicine.

A conventional needle-free injection system 10 is shown in FIG. 1, mainly includes a pressure accumulator 11, an output pipe 15 and a hand-holding device 17. The output pipe 15 is fixed at the front end of the pressure accumulator 11, and the hand-holding device 17 is fixed at the bottom end of the pressure accumulator 11. The pressure accumulator 11 can be connected to a fluid supply device 19. The fluid supply device 19 includes a gas cylinder 191, a pressure regulator 193 and a gas delivery pipe 195. By adjusting the pressure regulator 193, a high-pressure gas in the gas cylinder 191 can be controlled to be transmitted to the pressure accumulator 11 through the gas delivery pipe 195.

The output pipe 15 is provided with a charging hole 159, and a delivery material (not shown) is put into the output pipe 15 through the charging hole 159. The high-pressure gas will be accelerated when passing through the output pipe 15, and will drive the delivery material in the output pipe 15 to be injected into a target object (cell body).

The needle-free injection systems can utilize a gunpowder explosion, a high voltage, or a high-pressure gas as a power source to accelerate metal particles or solutions that include a delivery material. The structure of the conventional needle-free injection system is very complicated, which not only increases the cost and difficulty of production, but also may affect the gene transfer effect due to the poor production quality of the needle-free injection system.

### SUMMARY

It is a primary object of the present invention to provide a needle-free injection system, which can quickly connect or disassemble the output pipe and the pressure accumulator through an adapter tube. Through the adapter tube, the disadvantage of the output tube being loose during use can be avoided, and the convenience during use can be improved.

It is another object of the present invention to provide a needle-free injection system, which can fix the control panel on the outer surface of the pressure accumulator, so that the user can adjust a delivery procedure for putting the delivery material into the output pipe at any time, which can include Calibration, positioning, delivery time, number of deliveries and/or number of deliveries, thus improving ease of use.

It is another object of the present invention to provide a needle-free injection system. A touch control panel is fixed on the outer surface of the pressure accumulator, so as to abandon a physical wire to connect an external controller, so the needle-free injection system can be reduce its usage space and placement space.

It is another object of the present invention to provide a needle-free injection system, which will use the two ends of an adapter tube to connect the pressure accumulator and the output pipe individually, thereby not only improving the convenience of manufacture, but also reducing the turbulence flow in the output pipe and improve the effect of gene transfer.

It is another object of the present invention to provide a needle-free injection system, which can inject a proliferative agent (delivery material) into a treatment site of a patient at high speed without using an injection needle for acupuncture. The invention can relieve pain and discomfort of patients during treatment.

To achieve the above objective, the present invention provides a needle-free injection system, comprising: a pressure accumulator, one end of which is provided with an output portion; an output pipe, includes a connecting port, an ejection port and at least one connecting convex portion, wherein said connecting port and said ejection port are respectively located at two ends of said output pipe, and an accommodating space is formed between said connecting port and said ejection port, a dividing unit is arranged in said accommodating space, and at least one neck channel is arranged inside said dividing unit or between said dividing unit and an inner wall of said output pipe, said connecting convex portion is located on the outer surface of said output pipe, the outer surface of said output pipe is provided with a charging hole, which can be connected to said accommodating space; an adapter tube, includes a first connecting end, a second connecting end and at least one engaging groove, said first connecting end and said second connecting end are respectively located at two ends of said adapter tube, said engaging groove is located on said adapter tube, said first connection end can be sleeved on said output portion of said pressure accumulator, said connection port of said output pipe can enter said adapter tube from said second connection end and be connected to said output portion of said pressure accumulator, said connecting convex portion of said output pipe can be engaged with said engaging groove of said adapter tube; and a hand-holding device, fixed on the bottom end or the outer surface of said pressure accumulator, includes a shooting button for controlling a high-pressure gas entering or leaving said pressure accumulator.

To achieve the above objective, the present invention provides a needle-free injection system, comprising: a pressure accumulator, comprising an output portion and a power controller, said output portion is arranged at one end of said pressure accumulator, said power controller can control a power source to leave said pressure accumulator from said output portion; an output pipe, the two ends of said output pipe are respectively a connection port and an ejection port, an accommodating space is formed between said connection port and said ejection port, said connection port is connected to said output portion of said pressure accumulator, the outer surface of said output pipe is provided with a charging hole that can communicate with said accommodating space, and a dividing unit is arranged in said accommodating space; a delivery device, located at the top side of said pressure accumulator, has a driving device, a material storage member and a material delivery pipe, said material delivery pipe is arranged at the front end of the material storage member and communicated with said charging hole of said output pipe, said material storage member is used for placing a proliferating agent or a delivery material, said driving device can move forward and backward, thereby pushes said proliferating agent or said delivery material in said material storage member through said material delivery pipe and said charging hole into said accommodation space; a shooting button, connected to said power controller of said pressure accumulator; and a control panel, has a display and at least one control key, said control panel is electrically connected to the delivery device.

To achieve the above objective, the present invention provides a needle-free injection system, comprising: a pressure accumulator, one end of which is provided with an output portion; an output pipe, the two ends of which are respectively a connection port and an ejection port, an accommodating space is formed between said connection port and said ejection port, said connection port is connected to said output portion of said pressure accumulator, and the outer surface of said output pipe is provided with a charging hole that can be connected to said accommodating space; a shooting button, which is connected to said pressure accumulator to control a high-pressure gas entering or leaving said pressure accumulator; a delivery device, comprising a material storage member and a material delivery pipe, said material storage member is used for storing a delivery material, said material delivery pipe is connected to said charging hole of said output pipe; and a control panel, fixed on the outer surface of said pressure accumulator, the outer surface of said output pipe, or a portable control panel, said control panel has a display and at least one control key, said control panel is electrically connected to said delivery device.

In one embodiment of the present invention, wherein said dividing unit of said output tube has a first surface and a second surface, said first surface having a convex shape faces said connection port, and said second surface faces said ejection port, the distance between said first surface and said inner wall is gradually reduced from said connection port to said ejection port, the distance between said second surface and said inner wall are gradually increases from said connection port to said ejection port.

In one embodiment of the present invention, wherein said pressure accumulator includes a pressure trimmer, said pressure accumulator can be connected to a fluid supply device, said fluid supply device provides said high-pressure gas and leads said high-pressure gas into said pressure accumulator.

In one embodiment of the present invention, wherein said output portion of said pressure accumulator is provided with at least one fixing groove, said adapter tube is provided with a fixing hole corresponding to the position of said fixing groove, and a fixing member can pass through said fixing hole, so that said pressure accumulator is fixed on said adapter tube, said pressure accumulator is provided with a second locking hole adjacent to said output portion, said adapter tube is provided with a first locking hole corresponding to said second locking hole, and a locking piece can pass through said first locking hole and be fixed to said second locking hole.

In one embodiment of the present invention, wherein said pressure accumulator contains a solenoid valve, which is electrically connected to said shooting button and a power supply device, said pressure accumulator is connected to a control panel, which has a display and at least one control key.

In one embodiment of the present invention, wherein said control panel and said shooting button are fixed on the outer surface of said pressure accumulator or the outer surface of said output pipe.

In one embodiment of the present invention, wherein said control panel is a touch panel, said control key and said shooting button are an image control key and an image shooting key in said display.

In one embodiment of the present invention, wherein the bottom end or the outer casing of said pressure accumulator is fixedly provided with a hand-holding device, said shooting button is arranged on said hand-holding device.

In one embodiment of the present invention, wherein said pressure accumulator further comprises a pressure accumulating chamber, said pressure accumulator is connected to a power supply device, said power supply device provides said power, said power controller is located in said pressure accumulating chamber, said power controller is a solenoid valve.

In one embodiment of the present invention, wherein said output pipe is a jet engine type output pipe or a venturi type output pipe.

In one embodiment of the present invention, wherein said control panel is a touch panel, said control key is an image control key in said display, said shooting button is a physical button or an image shooting key in said display.

In one embodiment of the present invention, wherein said control panel is fixed to the top surface, the side surface, or the rear surface of said pressure accumulator, and the shooting button is provided on the outer surface of said pressure accumulator.

In one embodiment of the present invention, further comprising a hand-holding device fixed on the bottom end of said pressure accumulator, said shooting button is arranged on said hand-holding device, and said pressure accumulator is connected to a fluid supply device that provides said high-pressure gas for input to said pressure accumulator.

In one embodiment of the present invention, wherein said pressure accumulator is provided with a solenoid valve, said solenoid valve is electrically connected to said shooting button.

In one embodiment of the present invention, wherein said output pipe is a jet engine type output pipe or a venturi type output pipe.

In one embodiment of the present invention, further comprising an adapter tube, said adapter tube has a first connection end and a second connection end, said first connection end is used to connect and fix said pressure accumulator, and said second connection end is used to connect and fix said output pipe, so that said pressure accumulator is connected to said output pipe.

In one embodiment of the present invention, wherein said portable control panel comprises a wireless transmitter, said portable control panel is electrically connected to said delivery device by using said wireless transmitter, and the outer surface of said pressure accumulator is provided with a panel holder.

In one embodiment of the present invention, wherein said pressure accumulator is provided with a signal receiver, and said signal receiver can be electrically connected to said wireless transmitter of said control panel.

### BRIEF DESCRIPTION OF THE DRAWINGS

The structure as well as preferred modes of use, further objects, and advantages of this present disclosure will be best understood by referring to the following detailed description of some illustrative embodiments in conjunction with the accompanying drawings, in which:

FIG. 1 is a schematic diagram of a needle-free injection system in the prior art.

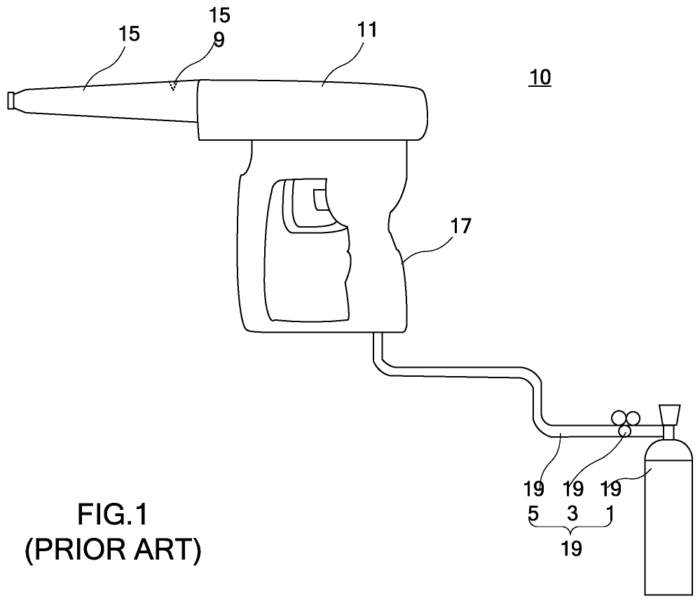

FIG. 2 is an exploded schematic diagram of an embodiment of the needle-free injection system of the present invention.

FIG. 3 is a side view of the needle-free injection system of the present invention.

FIG. 4 is a cross-sectional view of the output pipe of the present invention.

FIG. 5 is a side view of another embodiment of the needle-free injection system of the present invention.

FIG. 6 is a side view of another embodiment of the needle-free injection system of the present invention.

FIG. 7 is an exploded schematic diagram of another embodiment of the needle-free injection system of the present invention.

FIG. 8 is a side view of another embodiment of the needle-free injection system of the present invention.

FIG. 9 is a cross-sectional view of a venturi-type output pipe of the present invention.

FIG. 10 is a side view of another embodiment of the needle-free injection system of the present invention.

FIG. 11 is a side view of another embodiment of the needle-free injection system of the present invention.

FIG. 12 is a schematic diagram of another embodiment of the needle-free injection system of the present invention.

FIG. 13 is a side view of a preferred embodiment of the needle-free injection system of the present invention.

FIG. 14 is a schematic diagram of using of another embodiment of the needle-free injection system of the present invention.

FIG. 15 is a side view of another embodiment of the needle-free injection system of the present invention.

### DETAILED DESCRIPTION OF THE PREFERRED EMBODIMENTS

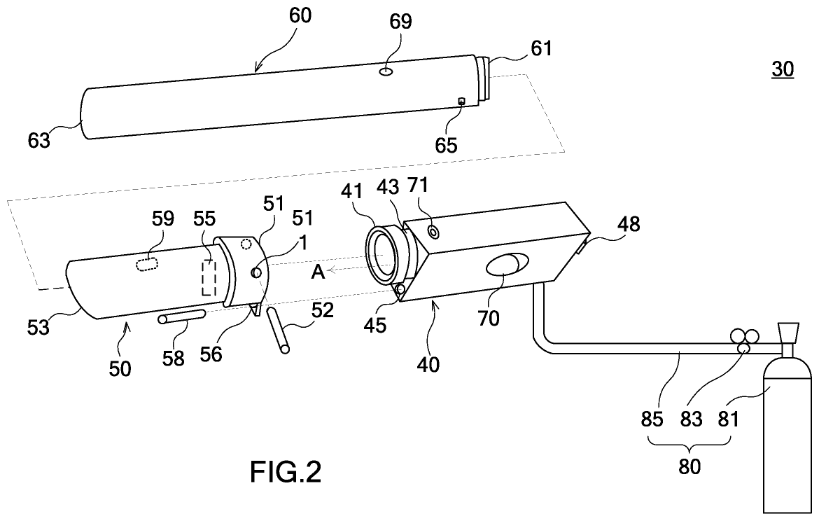

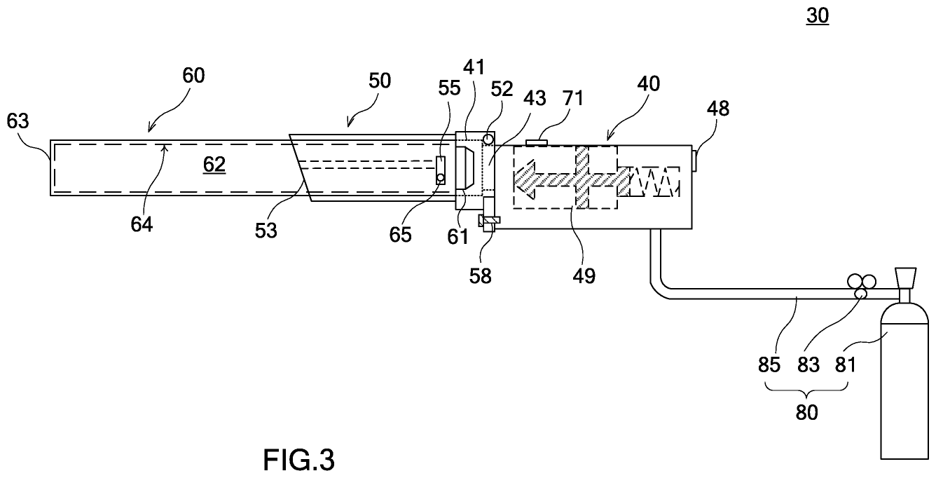

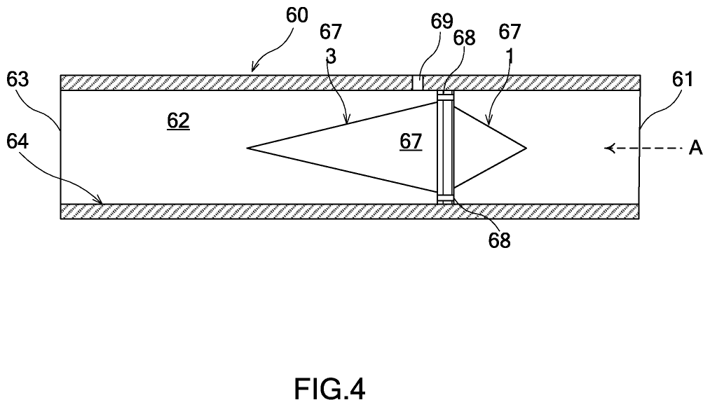

Referring to FIG. 2, FIG. 3 and FIG. 4, the needle-free injection system 30 of the present invention mainly includes a pressure accumulator 40, an adapter tube 50 and an output pipe 60. The two sides of the transfer pipe 50 are respectively connected to the pressure accumulator 40 and the output pipe 60. The pressure accumulator 40 includes an output portion 41, and outputs a power source A through the output portion 41. The power source A such as but not limited to a high-pressure gas. Specifically, the pressure accumulator 40 includes a pressure accumulating chamber and at least one valve, wherein the pressure accumulating chamber is connected to a fluid supply device 80 through the valve. When one of the valves is opened, the high-pressure gas A will be delivered to the pressure accumulator 40 by the fluid supply device 80, so that the gas pressure in the pressure accumulator 40 is greater than that of the outside world, for example, greater than one atmosphere. When another valve is opened, the high-pressure gas A in the pressure accumulator 40 can be output by the output portion 41 and leave the pressure accumulator 40.

The output pipe 60 includes a connecting port 61, an ejection port 63 and at least one connecting convex portion 65. The connection port 61 and the ejection port 63 are respectively located at two ends of the output pipe 60, and the connection convex portion 65 is located on an outer surface of the output pipe 60. The connection port 61 of the output pipe 60 is used to connect the output portion 41 of the pressure accumulator 40. The high-pressure gas A output from the output portion 41 of the pressure accumulator 40 will be introduced into the connection port 61 of the output pipe 60, and then pass through the ejection port 63 of the output pipe 60 is ejected to the outside. The high-pressure gas A will be accelerated in the process of passing through the output pipe 60, and the higher-speed gas will be ejected from the ejection port 63, which can also shoot a delivery material to a target object (eg, a cell body).

In an embodiment of the present invention, the output portion 41 has a concave part, and the connection port 61 has a convex part. When the connection port 61 is connected to the output portion 41, the convex part of the connection port 61 will be inserted into the concave part of the output portion 41, so as to achieve the purpose of the pressure accumulator 40 and the output pipe 60 being in close contact. A dividing unit 67 is disposed in the accommodating space 62, and the dividing unit 67 can be used to divide the accommodating space 62 into two connected spaces. The output pipe 60 of this embodiment is a jet engine type output pipe, and the dividing unit 67 can be a conical-shaped, a spindle-shaped or an olive-shaped object. The dividing unit 67 includes at least one first surface 671 and at least one second surface 673, and at least one neck channel 68 is disposed inside the dividing unit 67 or between the dividing unit 67 and the inner wall 64.

The first surface 671 with a convex configuration faces the connection port 61 of the output pipe 60. The space between the first surface 671 and the inner wall 64 of the output pipe 60 is formed to gradually decrease from the connection port 61 to the ejection port 63. In addition, the second surface 673 faces the ejection port 63 of the output pipe 60. The space between the second surface 673 and the inner wall 64 is formed to gradually increase from the connection port 61 to the ejection port 63. The speed of the high-pressure gas A can be accelerated through the jet engine type output pipe 60 of the present invention.

In another embodiment of the present invention, the second surface 673 can also be designed as a vertical plane configuration, which is different from the convex configuration shown in the figure. The second surface 673 of the vertical plane structure can also accelerate the speed of the high-pressure gas A input from the connection port 61 and ejected from the ejection port 63.

Since the dividing unit 67 of the present invention has the first surface 671 of the convex configuration, the high-pressure gas A can be guided to flow to the neck channel 68 close to the position of the inner wall 64 without being concentrated in the central area of the accommodating space 62. In other words, when the high-pressure gas A carries the solution or metal particles with the delivery material and shoots toward the ejection port 63, the solution or metal particles can be uniformly distributed over the entire cross-sectional area of the ejection port 63 instead of being concentrated in the central area. Therefore, the present invention can improve the transgenic effect of the delivery material, and can also reduce the damage caused by the high-pressure gas A to the target object.

The output pipe 60 further includes a charging hole 69, which can penetrate the inner wall 64 of the output pipe 60 and is connected to the accommodating space 62. The solution or metal particles with the delivery material can be fed into the accommodating space 62 through the charging hole 69. When the high-pressure gas A is input from the connection port 61 and passes through the output pipe 60, it will drive the delivery material in the accommodating space 62 to move toward the ejection port 63 and exit the needle-free injection system 30 from the ejection port 63.

The adapter tube 50 includes a first connecting end 51, a second connecting end 53 and at least one engaging groove 55. The first connection end 51 and the second connection end 53 are located at two ends of the adapter tube 50 respectively. The engaging groove 55 is located on an inner surface of the adapter tube 50 or forms a groove. The first connection end 51 of the adapter tube 50 can be sleeved on the output portion 41 of the pressure accumulator 40, and the second connection end 53 of the adapter tube 50 can be sleeved with the connection port 61 of the output pipe 60. Therefore, the adapter tube 50 can be connected to the pressure accumulator 40 and the output pipe 60 respectively.

In an embodiment of the present invention, a fixing groove 43 is provided on the output portion 41 of the pressure accumulator 40, and at least one fixing hole 511 is oppositely provided at the end of the adapter tube 50 close to the first connecting end 51. When the first connection end 51 of the adapter tube 50 is sleeved on the output portion 41 of the pressure accumulator 40, the fixing hole 511 of the adapter tube 50 will overlap with the fixing groove 43 of the pressure accumulator 40, and then a fixing piece 52 passes through the fixing hole 511 of the adapter tube 50 and is clamped in the fixing groove 43, thereby the pressure accumulator 40 can be fixed on the adapter tube 50.

The connection port 61 of the output pipe 60 is inserted into the second connection end 53 of the adapter tube 50, and the connection port 61 of the output tube 60 is connected to the output part 41 of the pressure accumulator 40 through the adapter tube 50. The connecting convex portion 65 of the output pipe 60 can be engaged in the engaging groove 55 of the adapter tube 50, so that the output pipe 60 can be fixed to the adapter tube 50. The connecting convex portion 65 and the engaging groove 55 may be one or more, and preferably two.

In an embodiment of the present invention, the connecting convex portion 65 of the output pipe 60 has a telescopic feature. When the output pipe 60 is inserted into the adapter tube 50, the connecting convex portion 65 can shrink inwardly due to the extrusion of the inner wall of the adapter tube 50. Once the connecting convex portion 65 reaches the position of the engaging groove 55 of the adapter tube 50, the connecting convex portion 65 can protrude again because it is no longer pressed by the inner wall of the adapter tube 50.

When the output pipe 60 is inserted into the adapter tube 50 from the second connection end 53, the charging hole 69 of the output pipe 60 may be blocked by the adapter tube 50, so a corresponding through hole 59 can be provided on the adapter tube 50. When the connecting convex portion 65 of the output pipe 60 is engaged with the engaging groove 55 of the adapter tube 50, the through hole 59 of the adapter tube 50 will be aligned with the charging hole 69 of the output pipe 60.

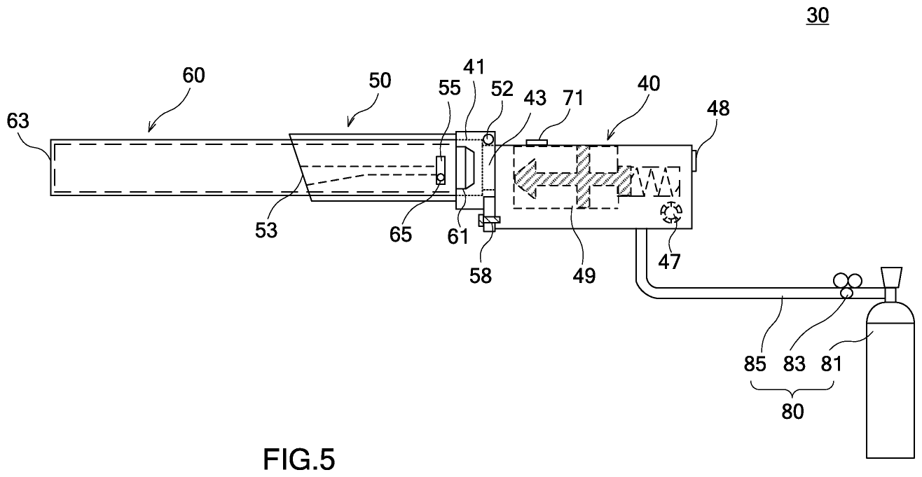

Of course, in another embodiment of the present invention, the length of the adapter tube 50 can also be reduced, so that when the output pipe 60 is connected to the adapter tube 50, the adapter tube 50 does not cover the charging hole 69 of the output pipe 60. In this way, the through hole 59 does not need to be provided on the adapter tube 50, as shown in FIG. 5.

In another embodiment of the present invention, the valve of the pressure accumulator 40 can be a solenoid valve 49, which is electrically connected to a power supply device 48, such as a battery, a mains socket or a power connection device. The plurality of valves of the solenoid valve 49 can control the high-pressure gas A entering and leaving the pressure accumulator 40. The fluid supply device 80 may be a powder explosion, high voltage or high-pressure gas as a power source. As shown in the figure, the fluid supply device 80 includes a gas cylinder 81, a pressure regulator 83 and a gas delivery pipe 85. The gas delivery pipe 85 is connected to the gas cylinder 81 and the pressure accumulator 40. The high-pressure gas A in the gas cylinder 81 can be input into the pressure accumulator 40 through the gas delivery pipe 85 by the pressure regulator 83.

In another embodiment of the present invention, a first locking hole 56 is provided at a position adjacent to the first connecting end 51 of the adapter tube 50, and a second locking hole 45 is provided at a corresponding position of the accumulator 40. When the adapter tube 50 fixes the pressure accumulator 40, a locking piece 58 passes through the first locking hole 56 and is locked to the second locking hole 45, thereby improving the fixing of the pressure accumulator 40 to the adapter tube 50 for stability.

In another embodiment of the present invention, a hand-holding device 70 and at least one shooting button 71 are provided on the outer surface of the pressure accumulator 40, so that the user can directly hold the needle-free injection system 30. By pressing the shooting button 71, the solenoid valve 49 is controlled, and the high-pressure gas A is guided into and out of the pressure accumulator 40, and is shot from the ejection port 63 together with the delivery material to the target object. The hand-holding device 70 may be the outer housing of the pressure accumulator 40.

Referring to FIG. 5, in an embodiment of the present invention, the engaging groove 55 can be designed to have a long guide groove or a hole groove that can extend to the second connecting end 53, thereby allowing the connecting convex portion 65 enters and exits the transfer tube 50. The output pipe 60 is rotated relative to the adapter tube 50, so that the connecting convex portion 65 of the output pipe 60 is engaged with the engaging groove 55 of the adapter tube 50.

Since the pressure regulator 83 of the fluid supply device 80 is relatively far from the pressure accumulator 40, it is inconvenient for the user of the needle-free injection system 30 to adjust the input speed and/or the input quantity of the high-pressure gas A at any time during use. Therefore, a pressure trimmer 47 may be provided on the pressure accumulator 40. The pressure trimmer 47 is provided on the pressure accumulator 40 and/or the hand-holding device 70. The pressure trimmer 47 is convenient for the user to adjust the input speed and/or the input quantity of the high-pressure gas A at any time, thereby improving the convenience and safety of the operation.

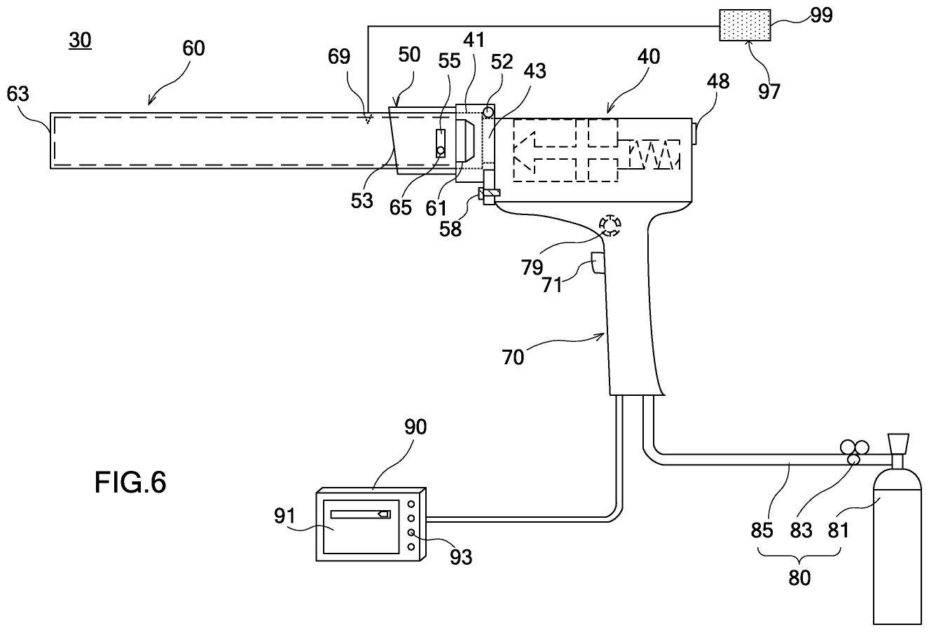

Referring to FIG. 6, the hand-holding device 70 of the present invention can also be designed in the shape of a handle and fixed on the bottom side of the pressure accumulator 40. Pressing the shooting button 71 of the hand-holding device 70 can control the high-pressure gas A to enter and exit the pressure accumulator 40, and let the delivery material (99) pass through the ejection port 63 to shoot at the target object. In addition, in order to avoid the wrong injection of the delivery material (99) due to the wrong pressing of the shooting button 71, the hand-holding device 70 can be provided with a safety switch 79. Of course, the number of times or the number of seconds of pressing the shooting button 71 can also be used as a safety switch (79) for whether to shoot the delivery material (99), so that the safety switch 79 can be replaced.

The present invention includes a delivery device 97 that stores a delivery material 99. The delivery device 97 is connected to the charging hole 69 of the output pipe 60, and the delivery device 97 can send the delivery material 99 into the accommodating space 62 of the output pipe 60 through the charging hole 69.

In an embodiment of the present invention, the pressure accumulator 40 is connected to a control panel 90, and the control panel 90 has a display 91 and at least one control key 95. The user can control the delivery time, delivery quantity or delivery times of the delivery material 99 entering the output pipe 60 through the control key 95. On the other hand, the display 91 can display the calibration, the positioning, the inventory of the delivery material, the delivery time, the delivery quantity and/or the projection times of the delivery device 97. The delivery device 97 can send the delivery material 99 into the accommodating space 62 of the output pipe 60 through the charging hole 69.

The delivery material 99 suitable for the needle-free injection system 30 of the present invention can be a hypodermic filler, a tissue promoter, a botulinum toxin, a biological substance, a medicine, a care product, a polymer particle, or a radiesse. For example but not limited to a DNA, RNA, protein, cosmetic composition, minerals, virus particles, Clostridium botulinum, hyaluronic acid, gelatinizer, collagen, vitamins, cellulose, fruit acids, transgenic organism, vaccine, essence, pearl, precious metals, pain-relieving genes and/or restore pigment.

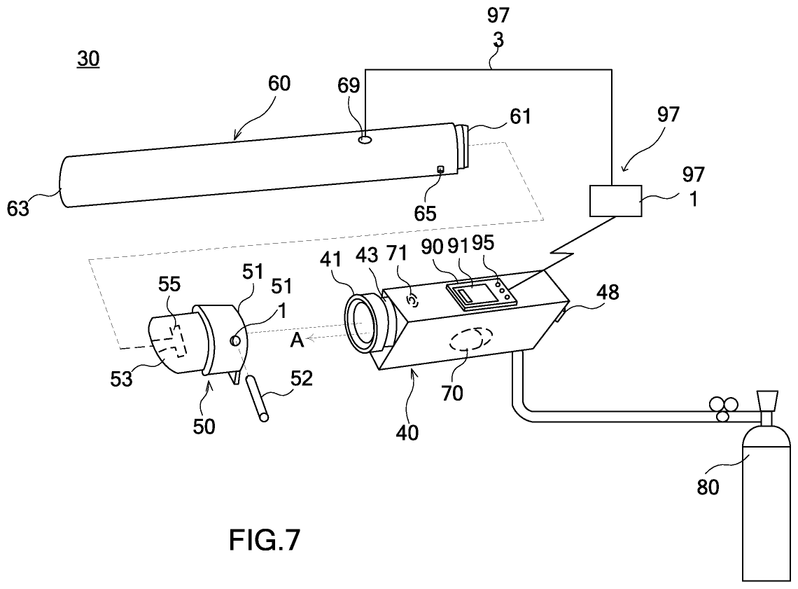

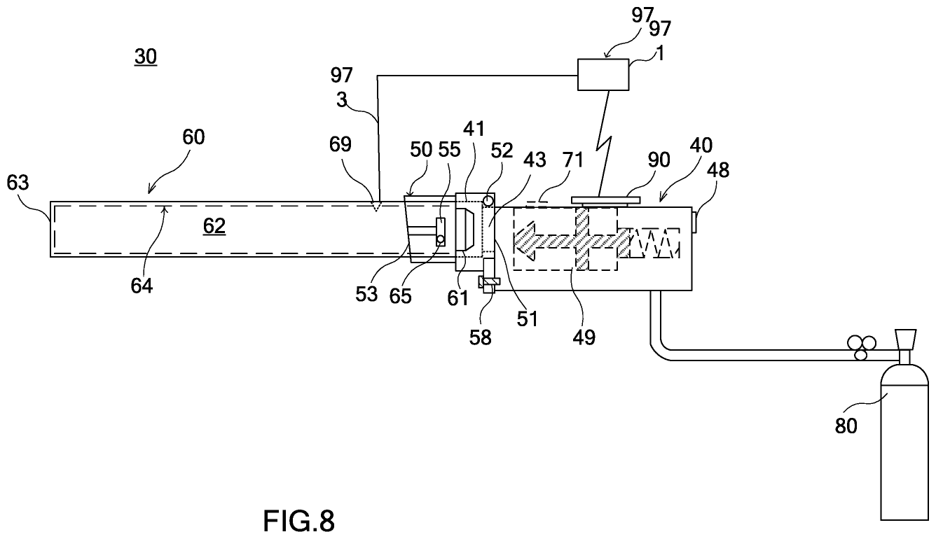

Furthermore, referring to FIGS. 7 and 8, the control panel 90 of the present invention will be fixed on the outer surface of the pressure accumulator 40. The delivery material can be placed in a material storage member 971 of a delivery device 97 and can be sent into the accommodating space 62 through a material delivery pipe 973 and the charging hole 69 of the output pipe 60. When the high-pressure gas A is input from the connection port 61 and passes through the output pipe 60, it will drive the delivery material in the accommodating space 62 to move toward the ejection port 63, and leave the ejection port 63 to eject toward the target object (cell body).

The control panel 90 of the present invention can be fixed on the outer surface of the pressure accumulator 40 by a snap method, a magnetic attraction method or a socket method. As shown in this figure, the control panel 90 is secured to the top surface of the pressure accumulator 40. The control panel 90 includes a display 91 and at least one control key 95, and is connected to the delivery device 97 through a physical wired or a wireless transmission. The user of the needle-free injection system 30 can control the delivery device 97 through the control keys 95 on the control panel 90 to make the delivery device 97 perform a delivery procedure, such as but not limited to a start-up, calibration, positioning, delivery time, delivery number and/or delivery times. The display 91 can display the data of the delivery process. Since the control panel 90 of the present invention can be fixed on the outer surface of the housing of the needle-free injection system 30, it can not only improve the convenience for the user to operate the needle-free injection system 30, but also reduce its use space or storage space.

In an embodiment of the present invention, the control keys 95 and the display 91 of the control panel 90 are independent physical components. The content of the command issued by the control key 95 can also be displayed on the display 91 to facilitate the user's operation.

In an embodiment of the present invention, the control panel 90 is a touch panel. The control keys 95 may be integrated into the display 91, and one or more image control keys 95 may be displayed on the display 91. The user can press the image control key 95 on the display 91 of the touch panel 90 to issue a delivery command. Of course, the shooting button 71 of the hand-holding device 70 can also be integrated on the control panel 90 to become the image shooting button 71 on the display 91 of the touch panel 90.

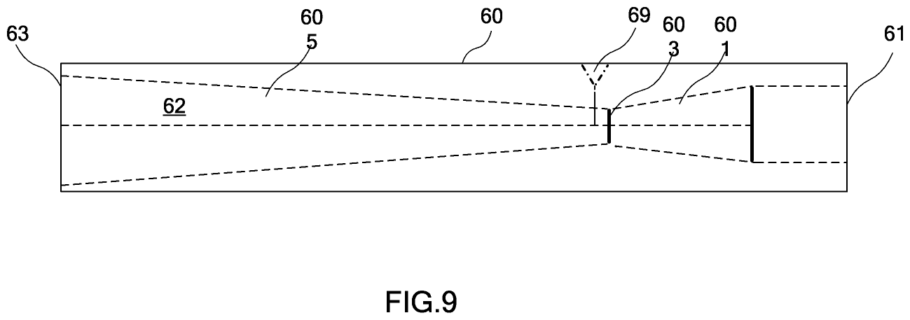

Referring to FIG. 9, the needle-free injection system 30 of the present invention can also be applied to a venturi type output tube 60. Two ends of the venturi type output pipe 60 are respectively a connecting port 61 and an ejection port 63, and there is an accommodating space 62 between the connecting port 61 and the ejecting port 63. A reducing portion 601, a throat portion 603 and a increasing portion 605 are respectively formed in the accommodating space 62. Similarly, a delivery material can be put into the increasing portion 605 through a charging hole 69 of the venturi-shaped output pipe 60.

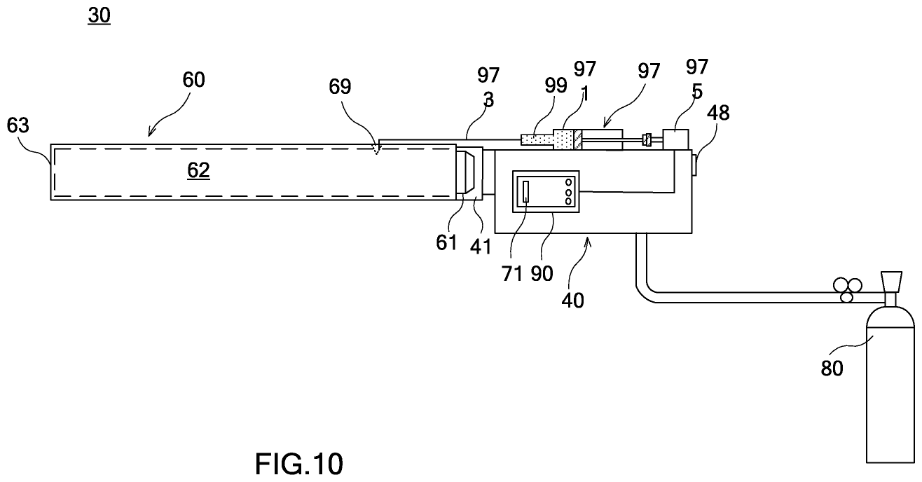

Referring to FIG. 10, the pressure accumulator 40 of the needle-free injection system 30 can be directly fitted with the output tube 60 without using the adapter tube 50. The connection port 61 of the output pipe 60 is sleeved on the output part 41 of the pressure accumulator 40, so that the output pipe 60 is connected to the pressure accumulator 40. The control panel 90 of the present invention can be fixed to the side surface of the housing of the pressure accumulator 40 by a magnetic attraction method, a screwing method, or a snapping method.

The delivery device 97 can be fixed on the top surface of the pressure accumulator 40. The material storage member 971 thereof can store a delivery material 99, and the material delivery pipe 973 is connected to the charging hole 69 of the output pipe 60. In the embodiment shown in the figure, the material storage member 971 of the delivery device 97 is in the form of a syringe, the material delivery pipe 973 is in the form of a needle. The rear end of the material storage member 971 is driven by a driving device 975 (such as a motor). By driving back and forth, the delivery material 99 stored in the material storage member 971 can be sequentially injected into the material delivery pipe 973, the charging hole 69 and the accommodating space 62 of the output pipe 60. The material storage member 971 and material delivery pipe 973 can be withdrawn upwards to leave the pressure accumulator 40, and the material storage member 971 can be refilled with the delivery material 99. The driving device 975 is electrically connected to the power supply device 48.

The delivery device 97 is electrically connected to the control panel 90, and receives a delivery instruction from the control panel 90 to perform procedures such as calibration, positioning, and delivery. The delivery device 97 can drive the delivery material 99 into the output pipe 60 for delivery time, delivery quantity or delivery times. The control panel 90 can also integrate the shooting button 71 to control the high-pressure gas A entering and leaving the pressure accumulator 40 and the output pipe 60. The shooting button 71 can be a physical button or an image shooting key on the touch panel (90).

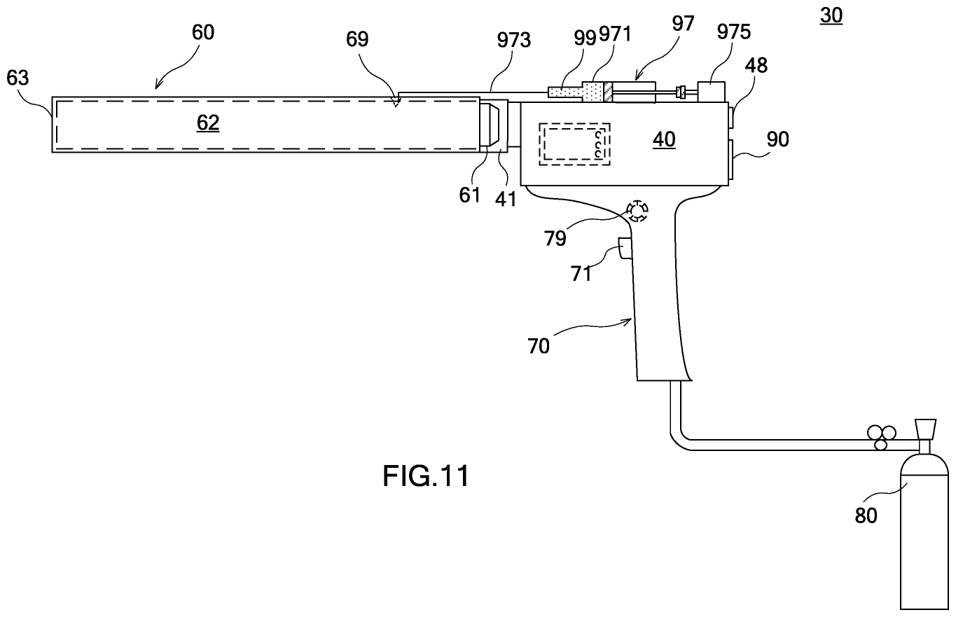

Referring to FIG. 11, the hand-holding device 70 of the needle-free injection system 30 of the present invention can be designed as a handle structure, and is arranged on the bottom surface of the pressure accumulator 40 by a magnetic attraction element, a snap element or other fixing means. The control panel 90 is fixed on the rear surface of the pressure accumulator 40 and is electrically connected to the delivery device 97. The control panel 90 may have a built-in battery, or be electrically connected to the power supply device 48. If the control panel 90 is a detachable device, it can be fixed to the pressure accumulator 40, the output pipe 60 or the outer casing of the hand-holding device 70 by a magnetic attraction. The control panel 90 can transmit the delivery instruction to the delivery device 97 via wireless communication.

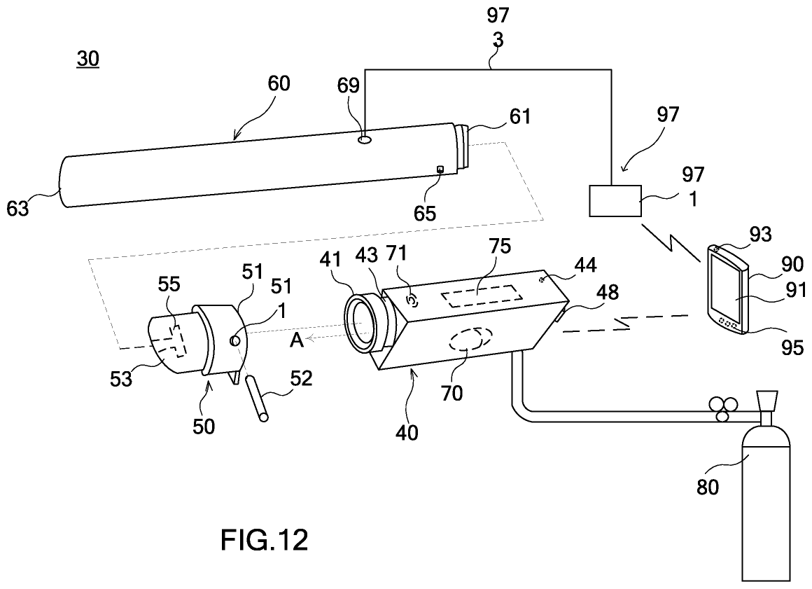

Referring to FIG. 12, the control panel 90 can be a portable control panel, such as but not limited to a mobile phone, a tablet computer or a digital media player (eg, iPod). The portable control panel 90 has a display 91, a wireless transmitter 93 and/or a control key 95. The control keys 95 may also be touch panel keys provided in the display 91. The wireless transmitter 93 can be a wireless network (wifi), a bluetooth or a ZigBee. The delivery instruction may be wirelessly transmitted to the delivery device 97 via the wireless transmitter 93. In this way, the user can directly use the user's mobile phone, tablet computer or digital media player as the control panel 90, which not only obtains the convenience of using the needle-free injection system 30, but also saves the cost of setting up the control panel 90.

In an embodiment of the present invention, one of the control keys 95 can also be designed as a shooting button (key) 71, and the firing command is transmitted to the pressure accumulator 40 through the wireless transmitter 93 of the control panel 90, and the high-pressure gas A enters and exits the pressure accumulator 40, the output pipe 60, and the delivery material 99 are ejected toward a target object (eg, a cell body) through the ejection port 63, thereby improving the convenience of use of the needle-free injection system 30. Of course, in this embodiment, the pressure accumulator 40 is also provided with a corresponding signal receiver 44, which can receive the firing command from the control panel 90 and/or the control key 95 in a wired or wireless manner.

In an embodiment of the present invention, the outer surface of the hand-holding device 70 or the outer surface of the pressure accumulator 40 is provided with a panel holder 75, such as but not limited to a magnet, a devil felt, a set of parts (such as leather cases) and/or an engaging part (such as buttons, hooks), etc. The portable control panel 90 can be fixed on the hand-holding device 70 or the pressure accumulator 40 through the panel holder 75, thereby improving the convenience for the user to operate.

The needle-free injection system 30 of the present invention can control the delivery material 99 to be injected into a target object through the control panel 90 and/or the fluid supply device 80, and can also control the depth of the delivery material 99 entering the target. For example, the delivery material 99 can be injected directly into the cell body or deep within the skin. Of course, it is also possible to control the delivery material 99 to only reach the skin surface, which can be the same as the external application effect of a general external application patch, and then absorb the diffusion effect or therapeutic effect of the delivery material 99 through the skin.

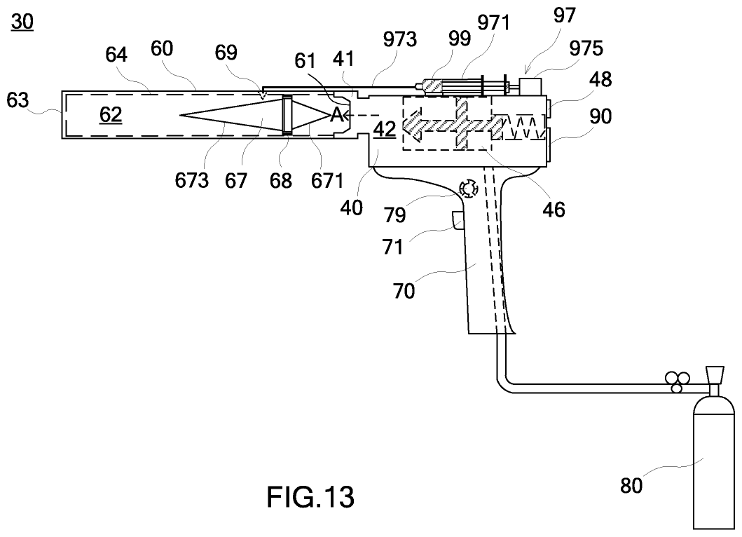

In addition, referring to FIG. 13, the needle-free injection system of the present invention can be applied to a proliferative device. As shown in the figure, the needle-free injection system 30 of the present invention mainly includes a pressure accumulator 40, an output pipe 60, a delivery device 97, a hand-holding device 70 and a power supply device 80. The pressure accumulator 40 has an output portion 41, a pressure accumulating chamber 42 and a power controller 46. The output portion 41 is located at the front end of the pressure accumulating chamber 42, and the power controller 46 is located in the pressure accumulating chamber 42. The pressure accumulator 40 is connected to the power supply device 80, and the power supply device 80 provides a power source A. The power source A can be a high-pressure gas, a compressed gas, a gunpowder detonating pressure and/or a high-pressure discharge. This figure takes a high-pressure gas as an example. The high-pressure gas A is input to the pressure accumulator 40 from the power supply device 80, so that the gas pressure in the pressure accumulator 40 is greater than that of the outside world, for example, greater than atmospheric pressure. The power controller 46 can control the power source A to enter and exit the pressure accumulator 40, and transmit the power source A to the output pipe 60 through the output portion 41.

The delivery device 97 includes a driving device 975, a material storage member 971 and a material delivery pipe 973, all of which can be fixed to the top surface of the pressure accumulator 40. A delivery material 99, such as a proliferative agent in this embodiment, can be placed in the material storage member 971, and the driving device 975 can move back and forth to push the delivery material 99 through the delivery pipe 973 and the charging hole 69 to put into the accommodating space 62 of the output pipe 60.

In an embodiment of the present invention, the material storage member 971 can be a syringe, the material delivery pipe 973 at the front end thereof can be connected to the charging hole 69 of the output pipe 60. The push rod at the rear end of the material storage member 971 contacts a driving device 975. The driving device 975 is a motor assembly that can move back and forth. When the driving device 975 is retracted, the material storage member 971 and/or the material delivery pipe 973 can be moved out and away from the delivery device 97, so that the storage member 971 and/or the proliferative agent 99 can be replaced. Conversely, when the driving device 975 moves forward, the push rod can be pushed forward, and the delivery material (eg, a proliferative agent) 99 placed in the material storage member 971 can be pushed into the accommodating space 62.

When the high-pressure gas A is input from the connection port 61 and passes through the output pipe 60, it will drive the delivery material (eg, a proliferative agent) 99 in the accommodating space 62 to move toward the ejection port 63, and is ejected from the ejection port 63 to a target object, such as tendons, ligaments, dermis, joint capsules, or cell bodies of an organism. The above-mentioned structure of the output pipe 60 is only a specific embodiment of the present invention, and is not intended to limit the scope of the right of the present invention.

In an embodiment of the present invention, a partition unit 67 is provided in the accommodating space 62 of the output pipe 60. The dividing unit 67 may divide the accommodating space 62 into two connected spaces. The output pipe 60 of this embodiment is a jet engine type output pipe, which can accelerate the speed of the power source A input from the connection port 61 and ejected from the ejection port 63.

In an embodiment of the present invention, the power controller 46 is a solenoid valve, and the pressure accumulating chamber 42 can be designed as an outer casing of the solenoid valve. In other words, the power controller 46 in the pressure accumulator 40 can be integrated with the pressure accumulating chamber 42, and the output portion 41 is located at the front end of the power controller 46. The driving device 975 and the power controller 46 can be electrically connected to a power supply device 48. The power supply device 48 can be a battery, a mains socket or a power connection device.

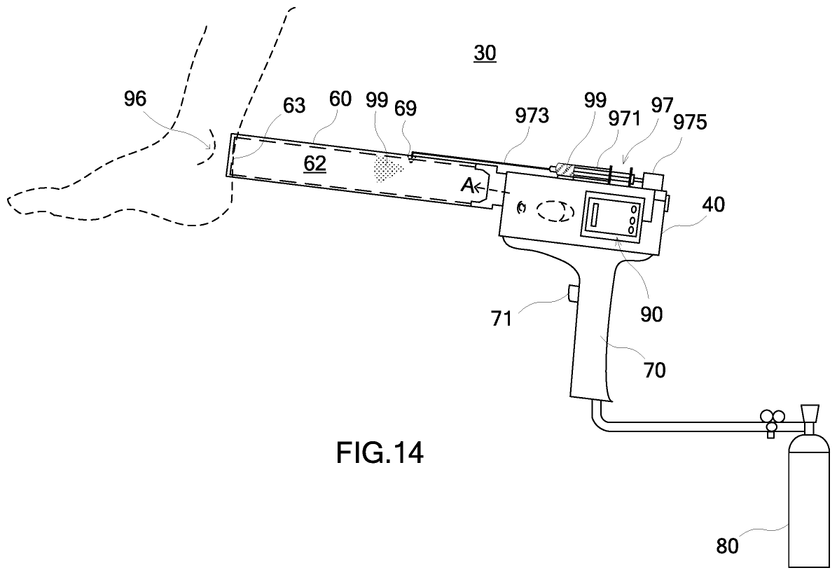

Referring to FIG. 14, a delivery material (eg, a proliferative agent) 99 will be placed in a material storage member 971 of the delivery device 97. The operator can set the delivery command of the delivery device 97 through the control panel 90, including but not limited to activation, calibration, positioning, delivery time, delivery quantity and/or delivery times. The power supply device 80 continuously provides a power A to the pressure accumulator 40. The operator can hold the hand-holding device 70, align the ejection port 63 of the output pipe 60 with the target object 96, and then press the shooting button 71 to control the power A and the proliferative agent 99 stored in the accommodating space 62 is injected into the target object 96, so that the proliferative agent 99 can be injected into the target object 96.

The needle-free injection system 30 of the present invention can change the position of the target object 96 to be injected continuously, in stages, and at any time. Therefore, the needle-free injection system 30 can abandon the injection needle of the traditional acupuncture method, and can easily deliver the delivery material (eg, proliferative agent) 99 is injected into the multiple target object 96, which not only improves the convenience of the operator during injection, but also reduces the discomfort and risk of the patient being punctured by the injection needle. The needle-free injection system 30 can further enhance the therapeutic effect of the delivery material 99.

The delivery material (eg, proliferative agent) 99 to which the needle-free injection system 30 of the present invention is applicable can be a cellular preparation, an osmotic shock, an irritant, or a chemotactic agent. More specifically, the proliferative agent 99 can be a high concentration platelet rich plasma (PRP), stem cells, glucose, glycerol, zinc sulfate, phenol, tannin, guaiacol, pumice, cod liver oleic acid, extracts, ozone and/or vitamins. The target object 96 to be hit is in a tendon, ligament, dermis, joint capsule, cell body, scalp tissue, skin and other tissues. It can also be repaired for various spinal joints or skeletal muscles and other painful parts. For sports injuries, Osteoarthritis, Shoulder and Neck Pain, Fifty Shoulders, Tennis Elbow, Elbow Pain, Wrist Pain, Knee Pain, Ankle, Spine Pain, Lower Back Pain, Plantar Fasciitis, Meniscus Injury, Cruciate Ligament Injury Rotator Tendon It has significant therapeutic effects on inflammation, tendinopathy, chronic pain, skin scars, firming and smoothing of skin, hyperplasia of scalp hair follicles, and prevention of hair loss.

The delivery material (eg, proliferative agent) 99 to which the needle-free injection system 30 of the present invention is applicable can be a cellular preparation, an osmotic shock, an irritant, or a chemotactic agent. More specifically, the proliferative agent 99 can be a high concentration platelet rich plasma (PRP), stem cells, glucose, glycerol, zinc sulfate, phenol, tannin, guaiacol, pumice, cod liver oleic acid, extract. substances, ozone and/or vitamins. The target 99 to be hit is a tendon, ligament, dermis, joint capsule, cell body, scalp tissue, skin, vertebral joint, skeletal muscle, etc. It is also suitable for sports injuries, degenerative arthritis, shoulder and neck pain, fifty shoulders, tennis elbow, elbow pain, wrist pain, knee pain, ankle pain, spine pain, lower back pain, plantar fasciitis, meniscus injury, cruciate ligament injury, rotator tendonitis, tendinopathy, chronic pain, skin scars, firming skin smooth, scalp hair follicle hyperplasia, preventing hair loss, etc. have significant therapeutic effects.

The needle-free injection system 30 of the present invention has a particularly significant therapeutic effect on platelet rich plasma (PRP). Of course, the collection of the delivery material (eg, proliferative agent) 99 still needs to go through procedures such as blood drawing of the patient body, blood separation by centrifuge, extraction of high-concentration platelet plasma is placed in the material storage member 971 of the delivery device 97, rather than a traditional injection needle, so the subsequent injection methods are completely different, and the effects after injection are also different.

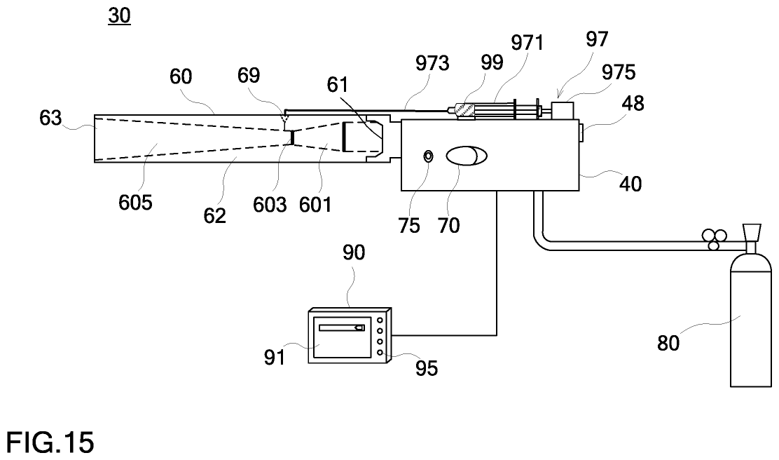

Finally, referring to FIG. 15, the needle-free injection system 30 of the present invention can be applied to a venturi-type output tube 60. The two ends of the venturi-shaped output pipe 60 are respectively a connecting port 61 and a jetting port 63, and a partition unit is arranged in the accommodating space 62, including a throat portion 603, the two ends of the throat portion 603 are respectively a reducing portion 601 and a increasing portion 605. A delivery material 99 can be fed into the increasing portion 605 through a charging hole 69 of the venturi-shaped output tube 60.

The above disclosure is only the preferred embodiment of the present disclosure, and not used for limiting the scope of the present disclosure. All equivalent variations and modifications on the basis of shapes, structures, features and spirits described in claims of the present disclosure should be included in the claims of the present disclosure.

What is claimed is:

1. A needle-free injection system, comprising:
a pressure accumulator, one end of which is provided with an output portion;
an output pipe, includes a connecting port, an ejection port and at least one connecting convex portion, wherein said connecting port and said ejection port are respectively located at two ends of said output pipe, and an accommodating space is formed between said connecting port and said ejection port, a dividing unit is arranged in said accommodating space, and at least one neck channel is arranged inside said dividing unit or between said dividing unit and an inner wall of said output pipe, said connecting convex portion is located on the outer surface of said output pipe, the outer surface of said output pipe is provided with a charging hole, which can be connected to said accommodating space;
an adapter tube, includes a first connecting end, a second connecting end and at least one engaging groove, said first connecting end and said second connecting end are respectively located at two ends of said adapter tube, said engaging groove is located on said adapter tube, said first connection end can be sleeved on said output portion of said pressure accumulator, said connection port of said output pipe can enter said adapter tube from said second connection end and be connected to said output portion of said pressure accumulator, said connecting convex portion of said output pipe can be engaged with said engaging groove of said adapter tube; and
a hand-holding device, fixed on the bottom end or the outer surface of said pressure accumulator, includes a shooting button for controlling a high-pressure gas entering or leaving said pressure accumulator.

2. The needle-free injection system according to claim 1, wherein said dividing unit of said output tube has a first surface and a second surface, said first surface having a convex shape faces said connection port, and said second surface faces said ejection port, the distance between said first surface and said inner wall is gradually reduced from said connection port to said ejection port, the distance between said second surface and said inner wall are gradually increases from said connection port to said ejection port.

3. The needle-free injection system according to claim 1, wherein said pressure accumulator includes a pressure trimmer, said pressure accumulator can be connected to a fluid supply device, said fluid supply device provides said high-pressure gas and leads said high-pressure gas into said pressure accumulator.

4. The needle-free injection system according to claim 1, wherein said output portion of said pressure accumulator is provided with at least one fixing groove, said adapter tube is provided with a fixing hole corresponding to the position of said fixing groove, and a fixing member can pass through said fixing hole, so that said pressure accumulator is fixed on said adapter tube, said pressure accumulator is provided with a second locking hole adjacent to said output portion, said adapter tube is provided with a first locking hole corresponding to said second locking hole, and a locking piece can pass through said first locking hole and be fixed to said second locking hole.

5. The needle-free injection system according to claim 1, wherein said pressure accumulator contains a solenoid valve, which is electrically connected to said shooting button and a power supply device, said pressure accumulator is connected to a control panel, which has a display and at least one control key.

6. A needle-free injection system, comprising:
a pressure accumulator, comprising an output portion and a power controller, said output portion is arranged at one end of said pressure accumulator, said power controller can control a power source to leave said pressure accumulator from said output portion;
an output pipe, the two ends of said output pipe are respectively a connection port and an ejection port, an accommodating space is formed between said connection port and said ejection port, said connection port is connected to said output portion of said pressure accumulator, the outer surface of said output pipe is provided with a charging hole that can communicate with said accommodating space, and a dividing unit is arranged in said accommodating space;
a delivery device, located at the top side of said pressure accumulator, has a driving device, a material storage member and a material delivery pipe, said material delivery pipe is arranged at the front end of the material storage member and communicated with said charging hole of said output pipe, said material storage member is used for placing a proliferating agent or a delivery material, said driving device can move forward and backward, thereby pushes said proliferating agent or said delivery material in said material storage member through said material delivery pipe and said charging hole into said accommodation space;
a shooting button, connected to said power controller of said pressure accumulator; and
a control panel, has a display and at least one control key, said control panel is electrically connected to the delivery device.

7. The needle-free injection system according to claim 6, wherein said control panel and said shooting button are fixed on the outer surface of said pressure accumulator or the outer surface of said output pipe.

8. The needle-free injection system according to claim 6, wherein said control panel is a touch panel, said control key and said shooting button are an image control key and an image shooting key in said display.

9. The needle-free injection system according to claim 6, wherein the bottom end or the outer casing of said pressure accumulator is fixedly provided with a hand-holding device, said shooting button is arranged on said hand-holding device.

10. The needle-free injection system according to claim 6, wherein said pressure accumulator further comprises a pressure accumulating chamber, said pressure accumulator is connected to a power supply device, said power supply device provides said power, said power controller is located in said pressure accumulating chamber, said power controller is a solenoid valve.

11. The needle-free injection system according to claim 6, wherein said output pipe is a jet engine type output pipe or a venturi type output pipe.

12. A needle-free injection system, comprising:
a pressure accumulator, one end of which is provided with an output portion;
an output pipe, the two ends of which are respectively a connection port and an ejection port, an accommodating space is formed between said connection port and said ejection port, said connection port is connected to said output portion of said pressure accumulator, and the outer surface of said output pipe is provided with a charging hole that can be connected to said accommodating space;
a shooting button, which is connected to said pressure accumulator to control a high-pressure gas entering or leaving said pressure accumulator;
a delivery device, comprising a material storage member and a material delivery pipe, said material storage member is used for storing a delivery material, said material delivery pipe is connected to said charging hole of said output pipe; and
a control panel, fixed on the outer surface of said pressure accumulator, the outer surface of said output pipe, or a portable control panel, said control panel has a display and at least one control key, said control panel is electrically connected to said delivery device.

13. The needle-free injection system according to claim 12, wherein said control panel is a touch panel, said control key is an image control key in said display, said shooting button is a physical button or an image shooting key in said display.

14. The needle-free injection system according to claim 12, wherein said control panel is fixed to the top surface, the side surface, or the rear surface of said pressure accumulator, and the shooting button is provided on the outer surface of said pressure accumulator.

15. The needle-free injection system according to claim 12, further comprising a hand-holding device fixed on the bottom end of said pressure accumulator, said shooting button is arranged on said hand-holding device, and said pressure accumulator is connected to a fluid supply device that provides said high-pressure gas for input to said pressure accumulator.

16. The needle-free injection system according to claim 12, wherein said pressure accumulator is provided with a solenoid valve, said solenoid valve is electrically connected to said shooting button.

17. The needle-free injection system according to claim 12, wherein said output pipe is a jet engine type output pipe or a venturi type output pipe.

18. The needle-free injection system according to claim 12, further comprising an adapter tube, said adapter tube has a first connection end and a second connection end, said first connection end is used to connect and fix said pressure accumulator, and said second connection end is used to connect and fix said output pipe, so that said pressure accumulator is connected to said output pipe.

19. The needle-free injection system according to claim 12, wherein said portable control panel comprises a wireless transmitter, said portable control panel is electrically connected to said delivery device by using said wireless transmitter, and the outer surface of said pressure accumulator is provided with a panel holder.

20. The needle-free injection system according to claim 12, wherein said pressure accumulator is provided with a signal receiver, and said signal receiver can be electrically connected to said wireless transmitter of said control panel.
